(19) United States
(12) Patent Application Publication
(10) Pub. No.: US 2015/0159185 A1
(43) Pub. Date: Jun. 11, 2015

(54) Intermittent application of reduced nitrogen sources for selection of PHB producing methanotrophs

(71) Applicant: The Board of Trustees of the Leland Stanford Junior University, Palo Alto, CA (US)

(72) Inventors: Craig S. Criddle, Redwood City, CA (US); Eric R. Sundstrom, Menlo Park, CA (US)

(21) Appl. No.: 14/404,527

(22) PCT Filed: May 29, 2013
(86) PCT No.: PCT/US2013/043069

§ 371 Date: Nov. 28, 2014

Related U.S. Application Data
(60) Provisional application No. 61/653,281, filed on May 30, 2012.

(51) Int. Cl. C12P 7/62

(52) U.S. Cl. CPC C12P 7/625

(57) ABSTRACT

A method of selection of polyhydroxybutyrate (PHB) producing Type II methanotrophs is provided that includes enriching PHB-producing methanotrophic strains, using a bioreactor, where ammonium includes a nitrogen source, where growth of the enriched PHB cells on ammonium selects for a PHB-producing methanotrophic strains by inhibiting survival of Type I organism growth, growing the enriched PHB-producing strains, using the bioreactor, on nitrate or urea to promote rapid and more dense growth of the enriched PHB-producing strains, where production of the PHB occurs when nitrogen is exhausted, and cycling between the enriching PHB-producing methanotrophic strains and growing the enriched PHB-producing strains, using the bioreactor, where a mixed culture of the PHB-producing strains are maintained without reducing growth rates of the methanotrophic strains or PHB production rates in the bioreactor.

### FIELD OF THE INVENTION

The invention relates to Poly(3-hydroxy)butyrate (PHB) production. More particularly, the invention relates to PHB production by methanotrophic organisms for production of high value polymers from a low cost feedstock of methane gas.

### BACKGROUND OF THE INVENTION

PHB production in methanotrophs is limited exclusively to strains in the type II group, having the genera methylocystis, mythylosinus, and methylocapsa. Under the mesophilic conditions most favorable for rapid growth of dense methanotrophic cultures, type I organisms tend to dominate, hindering long term study of mixed cultures. In the past, experiments have been conducted using pure cultures or known enrichment cultures in which type I organisms are deliberately excluded. While this strategy allows for short-term study, despite these efforts such cultures inevitably become contaminated in the longer term, usually within 2-4 weeks. In addition, use of pure and known mixed cultures limits biodiversity as compared to the use of highly diverse unknown mixed culture. This lack of genetic diversity adversely affects our ability to select for fast-growing strains capable of high PHB production. Establishment of a selective regime that effectively suppresses growth of type I organisms is also a critical to minimizing the cost of industrial scale production, due to the high costs associated with maintaining sterile reactor conditions.

Established selection techniques are currently incompatible with the goals of high growth and high PHB production. Selection techniques showing some degree of effectiveness include growth with no copper, growth in dilute medium, growth using gaseous nitrogen as the sole nitrogen source, and growth at low pH. All of these selection techniques suppress the growth of type I organisms at the expense of reduced growth in type II organisms. In addition, methods based on dilute media, low pH, or limitation of copper are difficult to implement even at bench scale, while nitrogen fixation is unreliable as a selector.

Selection based on nitrogen sources represents one possible alternative to these methods. Recent research on the genome for the type II organism Methylosinus trichosporium OB3b contains the pathway for hydroxylamine reduction to ammonium, while type I organisms appear incapable of reducing hydroxylamine and instead oxidize it to form nitrite, another toxic intermediate. Toxic hydroxylamine is produced as a byproduct of methane oxidation when ammonium is oxidized by the non-specific methane monooxygenase.

The ability to detoxify hydroxylamine by reduction to ammonium is a potential competitive advantage for type II organisms in ammonium rich environments. This hypothesis is borne out by the results of competition experiments in which the type II strain Methylocystis sp. strain ATCC 49242 outcompeted the type I strain Methylomicrobium album ATCC 33003 when grown with high levels of ammonium. Both ammonium and urea are inexpensive nitrogen sources that should produce higher yields than nitrate due to their reduced state.

What is needed is a method of using ammonium or urea for their ability to select for high densities of PHB producing type II organisms.

### SUMMARY OF THE INVENTION

To address the needs in the art, a method of selection of polyhydroxybutyrate (PHB) producing Type II methanotrophs is provided that includes enriching PHB-producing methanotrophic strains, using a bioreactor, with ammonium included as a nitrogen source, where growth of the enriched methanotrophic cells on ammonium selects for a PHB-producing methanotrophic strains by inhibiting survival of Type I organism growth, growing the enriched PHB-producing strains, using the bioreactor, on nitrate or urea to promote rapid and more dense growth of the enriched PHB-producing strains, where production of the PHB occurs when nitrogen is exhausted, and cycling between enriching PHB-producing methanotrophic strains and then growing the enriched PHB-producing strains, using the bioreactor, where a mixed culture of the PHB-producing strains are maintained without reducing growth rates of the methanotrophic strains or PHB production rates in the bioreactor.

According to one aspect of the invention, a reduced level of hydroxylamine relative to the ammonium is used instead of the ammonium to inhibit the growth of non-PHB producing organisms.

In another aspect of the invention, the time spent in the enriching step, the growth step, or the growth step and the enriching step is varied.

In a further aspect of the invention, additional nitrate or urea growth steps with reduced frequency of ammonium growth steps is provided.

In another aspect of the invention, a reduced level of hydroxylamine relative to the ammonium is used instead of the ammonium to inhibit the growth of non-PHB producing organisms.

According to another aspect of the invention, methane is limited during growth on the ammonium, where reduced methane levels increase ammonium toxicity and forces organisms to draw upon stored PHB, where and an increased PHB production during subsequent the cycles occurs.

In a further aspect of the invention, methane is limited during initial growth on the nitrate, where cells with stored PHB replicate to increase the PHB production during subsequent the cycles.

In yet another aspect of the invention the nitrogen sources are added incrementally throughout the enriching step or the growth step, where the nitrogen sources can include ammonium, nitrate, urea, or hydroxylamine.

According to a further aspect of the invention, the ammonium or hydroxylamine are added incrementally throughout multiple steps to increase selection for producers of the PHB.

In one aspect of the invention, the bioreactor includes a biofilm growth system, where pulses of ammonium addition are used to suppress growth of non-PHB producers in biofilm cultures.

### BRIEF DESCRIPTION OF THE DRAWINGS

FIGS. 1a-1c show fluorescence of methanotrophs grown in a known mixed culture of two type I and two type II organisms, according to one embodiment of the invention.

FIGS. 2a-2d show fluorescence of methanotrophs grown in enrichment culture inoculated with activated sludge, according to one embodiment of the invention.

FIGS. 3a-3b show total suspended solids (3a) and PHB production (3b) in cycling reactors over time, according to one embodiment of the invention.

FIGS. 4a-4b show cell concentrations and percent PHB by dry cell weight in two cycling bioreactors over the course of a single cycle, according to one embodiment of the invention.

FIG. 5 shows a schematic drawing of a bioreactor used in the selection of PHB producing Type II methanotrophs, according to one embodiment of the invention.

### DETAILED DESCRIPTION

Selection for dense cultures of PHB producing organisms from a diverse inoculum was until now a non-trivial unsolved problem, where type II organisms known to produce PHB are typically slower growing under mesophilic conditions most favorable to growth of methanotrophs. Reactors initially inoculated with PHB producing cultures are therefore unstable and highly susceptible to takeover, necessitating extensive and costly sterilization procedures. Using the selective regime according to the current invention, it is now possible to select for high PHB production without concern for sterility, thereby reducing capital costs, operating costs, and overall risk. In addition, use of more diverse cultures allow for the selection of more highly adapted PHB producing methanotrophs.

Methanotrophic bacteria naturally produce poly(3-hydroxy)butyrate (PHB) as a carbon and energy storage polymer. Production of PHB is contingent upon selection of PHB producing Type II methanotrophs, as opposed to type I methanotrophs, which are not known to produce PHB. The current invention provides a selection technique where cells are originally enriched with ammonium, hydroxylamine or the like, as the sole nitrogen source. In one embodiment, growth on ammonium selects for PHB producing methanotrophs by dramatically inhibiting the growth and survival of type I organisms. Because growth of type II methanotrophs is also slow on ammonium, the cells are then transferred to growth on nitrate or urea, both of which allow for rapid growth of dense cultures followed by PHB production when all nitrogen is exhausted. By cycling between multiple nitrogen sources it is possible to maintain a mixed culture of PHB producing methanotrophs without sacrificing density, growth rates, or total PHB production.

The current invention provides PHB as a high molecular weight carbon storage polymer produced by a wide variety of microorganisms and useful as a commercial thermoplastic. According to one embodiment, the invention provides PHB production by methanotrophic organisms for production of high value polymer from a low cost feedstock of methane gas. Here, production of PHB by methanotrophs grown in open systems is contingent upon selection of PHB producing organisms from a diverse inoculum. According to one embodiment, the current invention selects robustly for PHB producing methanotrophs while still allowing growth at high rates and densities.

According to one aspect of the invention, a reduced level of hydroxylamine relative to the ammonium is used instead of the ammonium to inhibit the growth of non-PHB producing organisms.

In another aspect of the invention, the time spent in the enriching step, the growth step, or the growth step and the enriching step is varied.

In a further aspect of the invention, additional nitrate or urea growth steps with reduced frequency of ammonium growth steps is provided.

In another aspect of the invention, a reduced level of hydroxylamine relative to the ammonium is used instead of the ammonium to inhibit the growth of non-PHB producing organisms.

According to another aspect of the invention, methane is limited during growth on the ammonium, where reduced methane levels increase ammonium toxicity and forces organisms to draw upon stored PHB, where and an increased PHB production during subsequent the cycles occurs.

In a further aspect of the invention, methane is limited during initial growth on the nitrate, where cells with stored PHB replicate to increase the PHB production during subsequent the cycles.

In yet another aspect of the invention the nitrogen sources are added incrementally throughout the enriching step or the growth step, where the nitrogen sources can include ammonium, nitrate, urea, or hydroxylamine.

According to a further aspect of the invention, the ammonium or hydroxylamine are added incrementally throughout multiple steps to increase selection for producers of the PHB.

In one aspect of the invention, the bio reactor includes a biofilm growth system, where pulses of ammonium addition are used to suppress growth of non-PHB producers in biofilm cultures.

In a further embodiment, the system is adapted to include multiple reactors, including smaller reactors for growth on ammonium and larger reactors for growth and PHB production with nitrate or urea as the nitrogen source. By cycling between multiple reactors capital costs are reduced while still preserving the cycling properties of the system.

Intermittent addition of ammonium according to the current invention is new, as compared to previous studies that attempt to select organisms using non-variable conditions. No cultures enriched under non-variable conditions have matched the success of intermittent ammonium addition.

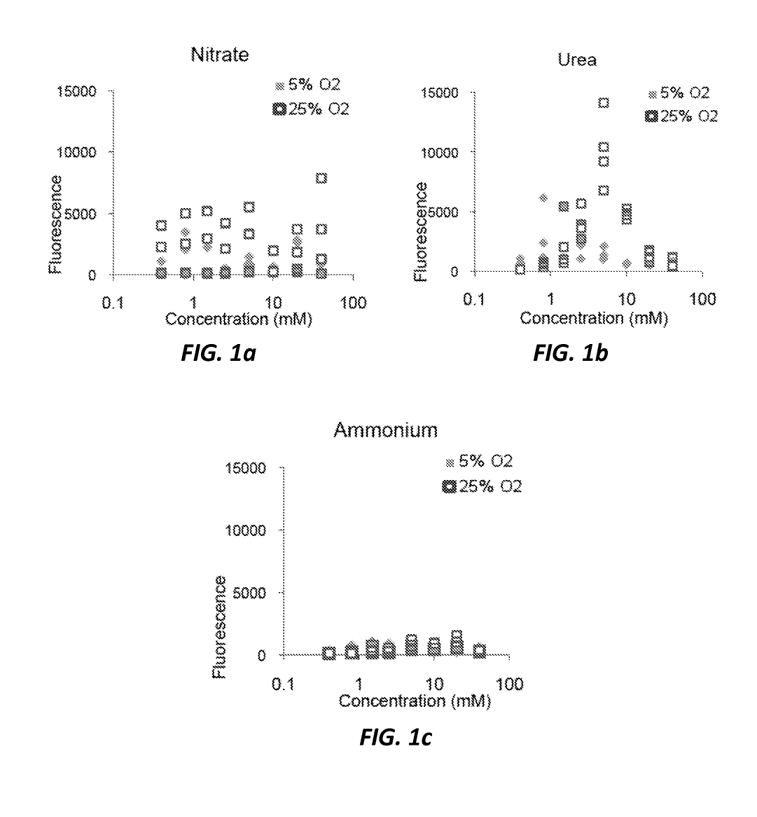

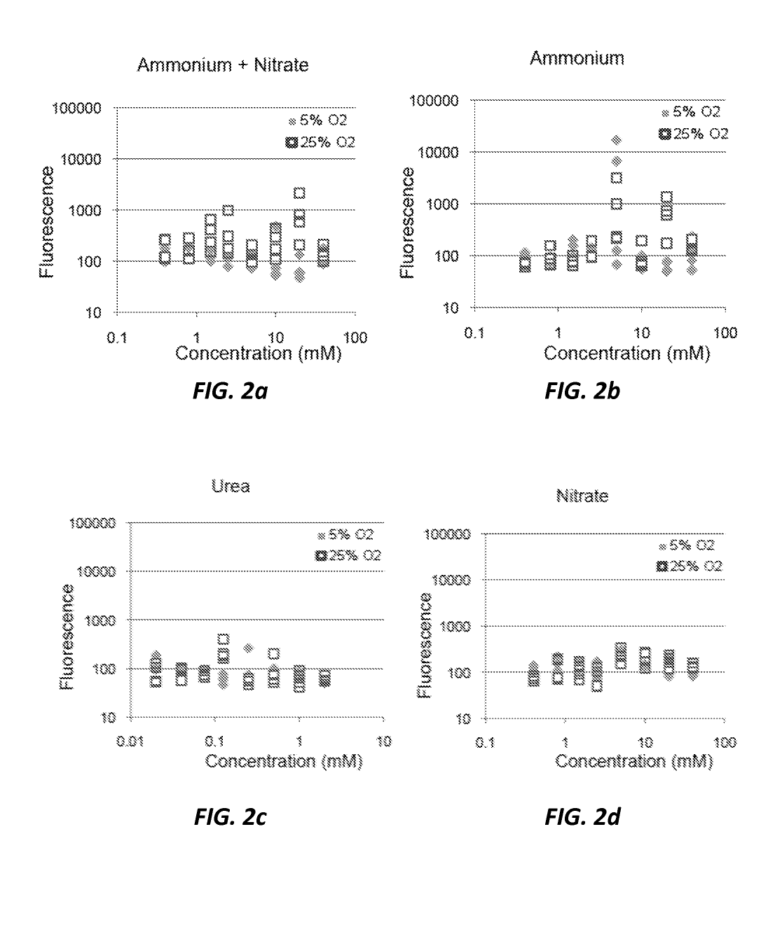

To determine the effectiveness of varying nitrogen sources as a selection mechanism according to the current invention, competition experiments were conducted using a known mixed culture of two type I and two type II strains (FIGS. 1a-1c) and an unknown enrichment from activated sludge (FIGS. 2a-2d). A high throughput growth and analysis system was employed to determine PHB production, with 8 nitrogen concentrations tested at two oxygen levels and 4 replicates per condition. In known mixed culture, growth on 5 mM urea and 25% oxygen was the most effective selector for PHB production, with mixed results for nitrate and little growth observed in cultures grown on ammonium.

Metabolism of urea requires cleavage into two molecules of ammonium and one molecule of carbon dioxide, and therefore growth on urea creates a slow supply of ammonium suitable for growth of type II methanotrophs while still inhibitory towards the type I cultures. Alternatively, the type I cultures in this experiment lacked the pathways necessary to process either urea or ammonium.

FIGS. 1a-1c show fluorescence graphs of methanotrophs grown in a known mixed culture of two type I and two type II organisms: Methylomicrobium album BG8, Methylomonas LW13, Methylocystis parvus OBBP, and Methylosinus trichosporium OB3b. Cultures were inoculated into 96 well microplates containing gradients of three nitrogen sources, nitrate, urea, and ammonium. Microplates were incubated for 10 days under a continuous flow of either 5% oxygen/95% methane or 25% oxygen/75% methane. Fluorescence was analyzed by high throughput flow cytometry after staining with Nile red dye.

Results differed substantially in cultures enriched from activated sludge, with the highest fluorescence observed in cultures grown with 5 mM ammonium. No elevated fluorescence was observed in cultures grown on urea as compared to nitrate. In cultures grown with varying levels of ammonium in addition to 10 mM nitrate, fluorescence was substantially elevated as compared to cultures grown on 10 mM nitrate alone, particularly at 2 mM ammonium and above.

FIGS. 2a-2d show fluorescence graphs of methanotrophs grown in enrichment culture inoculated with activated sludge. Cultures were inoculated into 96 well microplates containing gradients nitrate, urea, and ammonium. In addition, some ammonium replicates were amended with 10 mM nitrate. Microplates were incubated for 10 days under a continuous flow of either 5% oxygen/95% methane or 25% oxygen/75% methan. Fluorescence was analyzed by high throughput flow cytometry after staining with Nile red dye.

Due to the promising results but small volumes of the microplate enrichments, further enrichments were conducted in 4 L fermenters to more allow for more diversity in the inoculum and to more effectively quantify the resulting communities. Fermenters containing 10 mM of ammonium, urea, and nitrate were inoculated with activated sludge and allowed to reach plateau phase. The resulting cultures were analyzed for PHB content, biomass, and community composition. Cultures enriched with nitrate showed dense growth of up to ˜4 g/L but failed to produce measurable PHB (Table 1). Cultures grown on ammonium grew only to low densities up to ˜0.4 g/L, but did produce PHB in one replicate. Cultures enriched on urea showed both results, with one enrichment growing to high density while the remaining two struggled. While molecular analysis is in progress, a cursory visual analysis indicates that low density cultures were cloudy white, while high density cultures were a pink or orange color characteristic of type I methanotrophs. This analysis is consistent with the hypothesis of type II organisms in the low-density ammonium and urea cultures and type organisms in the high density urea and nitrate cultures.

Based on these results, it is apparent that growth on ammonium selects for type II methanotrophs but inhibits their growth and PHB production. Nitrate enrichments grew much more rapidly but failed to produce PHB. It is therefore beneficial to combine growth on ammonium with growth on nitrate, to both inhibit type II organisms and encourage rapid growth. Two regimes were tested based on this hypothesis, one in which ammonium and nitrate were applied simultaneously and one in which cells were enriched on ammonium and then amended with nitrate. 2 of the 3 enrichments grown with both nitrogen sources at once demonstrated PHB production, but at very low levels (Table 1). When the two nitrogen sources were applied in sequence, significantly higher PHB production was observed, particularly in the enrichment containing the highest level of ammonium. In this enrichment PHB production was measured at 10% in a dense culture of 2.85 g/L TSS.

TABLE 1
PHB production in enrichment culture.

| | Enrich- | Bio- | | Total |
|---|---|---|---|---|
| | ment | mass | Percent | PHB |
| Sample | number | (g/L) | PHB | (g/L) |
| 10 mM Ammonium | 1 | 0.36 | 3.1% | 0.01 |
| 10 mM Ammonium | 2 | 0.37 | 0.0% | 0.00 |
| 10 mM Ammonium | 3 | 0.27 | 0.0% | 0.00 |
| 10 mM Urea | 1 | 0.35 | 0.0% | 0.00 |
| 10 mM Urea | 2 | 0.11 | 0.0% | 0.00 |
| 10 mM Urea | 3 | 3.65 | 0.0% | 0.00 |
| 10 mM Nitrate | 1 | 1.51 | 0.0% | 0.00 |
| 10 mM Nitrate | 2 | 3.94 | 0.0% | 0.00 |
| 10 mM Nitrate | 3 | 3.30 | 0.0% | 0.00 |
| 1 mM ammonium + 10 mM nitrate | 4 | 1.87 | 1.8% | 0.03 |
| 4 mM ammonium + 10 mM nitrate | 4 | 1.43 | 1.5% | 0.02 |
| 10 mM ammonium + 10 mM nitrate | 4 | 0.39 | 0.0% | 0.00 |
| 2 mM ammonium | 5 | 0.32 | 3.2% | 0.01 |
| 5 mM ammonium | 5 | 0.21 | 0.0% | 0.00 |
| 10 mM ammonium | 5 | 0.35 | 0.0% | 0.00 |
| 2 mM ammonium −> 10 mM nitrate | 5 | 2.55 | 3.1% | 0.08 |
| 5 mM ammonium −> 10 mM nitrate | 5 | 3.77 | 3.0% | 0.11 |
| 10 mM ammonium −> 10 mM nitrate | 5 | 2.85 | 10.0% | 0.28 |

Table 1 shows PHB production in enrichment culture grown in 4 L bioreactors under a continuous flow of 25% oxygen and 75% methane. The enrichment number refers to the batch activated sludge used for each replicate. All the reactors were grown for 10 days and harvested after reaching plateau phase. Reactors that were inoculated in enrichment five were grown on ammonium only, the diluted 3:1 with fresh media and allowed to grow for 4 days with 10 mM nitrate.

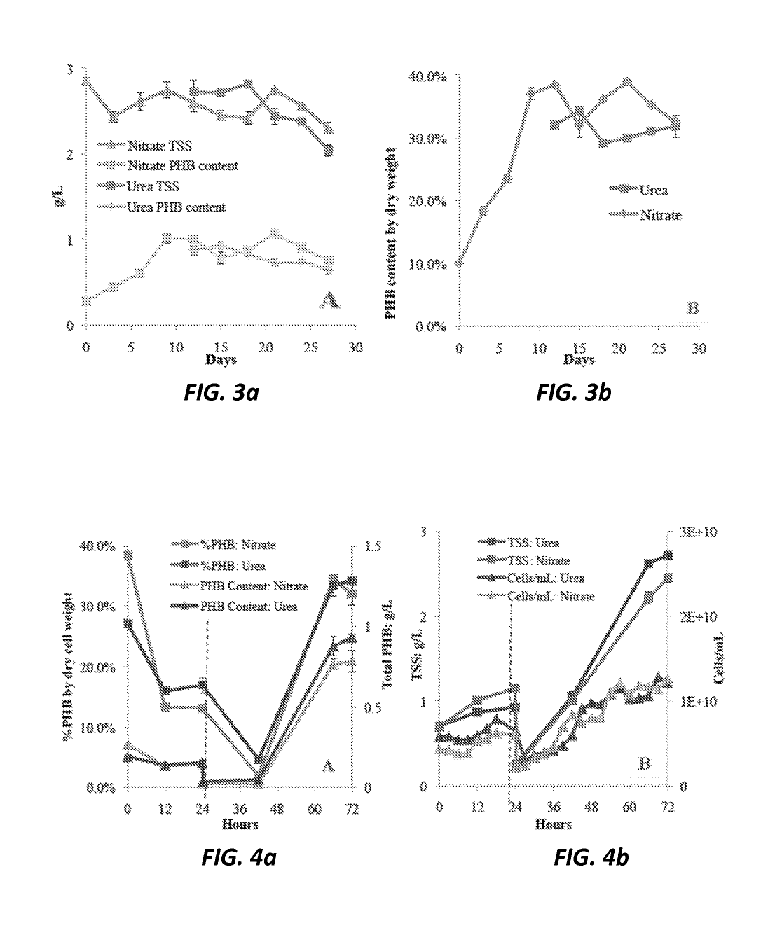

Based on the success of sequential growth on ammonium and nitrate, the 10 mM ammonium ->10 mM nitrate reactor was left in operation, cycling between one day of growth on ammonium and two days of growth on nitrate. After 4 cycles, a new reactor was inoculated from the original, this one cycling between ammonium and urea. Both reactors were successful at selecting and maintaining a dense, PHB producing culture (FIGS. 3a-3b). In the nitrate cycling reactor, PHB production rose from 10% initially to a high of 39%, with a maximum of 1.07 g/L PHB produced at the end of cycle. The urea reactor was similarly productive, producing up to 34% PHB and 0.93 g/L PHB. The nitrate cycling reactor was allowed to run for a full month with no indication of contamination.

FIGS. 3a-3b show total suspended solids (3a) and PHB production (3b) in cycling reactors over time. Both reactors were operated on a 3-day cycle in which cells were diluted 3:1 and grown on 10 mM ammonium for 24 hours, then diluted 3:1 again and grown on 8 mM nitrate or 4 mM urea for 48 hours. The urea cycling reactor was inoculated from the nitrate reactor after 4 cycles.

Looking more closely within each cycle, growth is slow during the ammonium phase, with only a slight increase in cell concentration and total suspended solids (FIG. 4b). Simultaneously, within the first 12 hours of ammonium exposure both PHB percentage by dry weight and total PHB levels declined significantly in both the urea and nitrate reactors. After the addition of nitrate, replication begins almost immediately in both reactors and continues at an exponential pace for 18 hours, after which cell growth tapers and PHB production begins due to exhaustion of available nitrogen sources. Growth on nitrate does not display the lag phase present in previous experiments, indicating that growth on ammonium was sufficient to replenish nitrogen levels and begin replication. It is likely that during growth on ammonium the PHB consumed was used as a source of reducing power for the reduction of hydroxylamine to ammonium, indicating a potentially unobserved use for stored PHB in methanotrophic organisms. Overall behavior in both reactors was remarkably similar, and urea is therefore a preferable nitrogen source do to its mild selective properties, low cost, and potential for higher yields through use of a reduced nitrogen source, according to one embodiment of the invention.

FIGS. 4a-4b show cell concentrations and percent PHB by dry cell weight in two cycling bioreactors over the course of a single cycle. PHB content was measured by flow cytometry and calibrated using gas chromatography. The ammonium phase for both reactors is sown left of the dashed line, while the nitrate/urea phase is to the right.

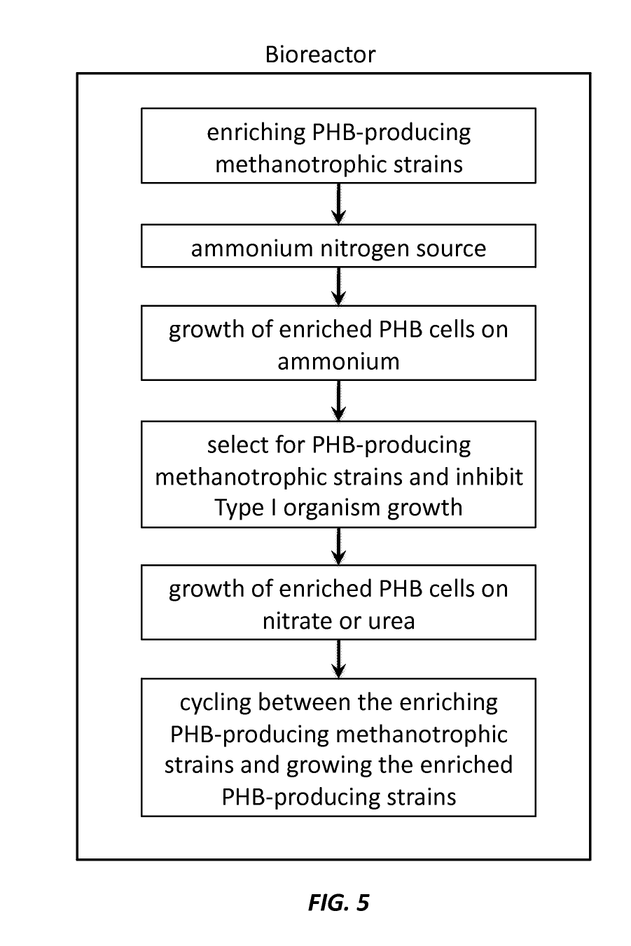

FIG. 5 shows a schematic drawing of a bioreactor used in the selection of PHB producing Type II methanotrophs, according to one embodiment of the invention.

By combining methane limitation with the type II selection described above, it is possible to achieve further increases in PHB production. In particular, methane limitation may be effective during growth on ammonium, during which low levels of methane would encourage increased production of hydroxylamine while simultaneously reducing the amount of methane available for hydroxylamine reduction. Cells with high levels of PHB use their stored reducing power to survive and reproduce in this harsh environment, while cells lacking adequate PHB may be unable to reproduce.

Establishment of an effective selective regime according to the current invention is a critical breakthrough with regard to the economical production of PHB by methanotrophs. These reactors require no precautions to prevent contamination, and should instead incorporate outside species into a more efficient community. Such a system can vastly reduce capital costs by eliminating the need for sterilization, and could be operated at small scale by an inexperience operator. As an example, the nitrate/ammonium cycling bioreactor described above continued stable operation beyond 30 days of operation. With PHB content stabilizing at 0.8-1.1 g/L and 3 L of culture produced every 3 days per bioreactor, production is at least 1.6-2.2 grams per day of PHB in the two reactors.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example the duration of periods of growth on ammonium and periods of growth on ammonium or nitrate may be varied to maximize selection for PHB producing organisms or to maximize growth and PHB production. Multiple periods of growth with urea and nitrate as nitrogen sources may be used consecutively with intervening phases of growth with ammonium nitrogen sources. Hydroxylamine may be substituted for ammonium to provide selection for Type II methanotrophs while allowing more control over reactor conditions. Cycling of nitrogen sources could be applied to other reactor systems, including fluidized bed reactors, membrane bioreactors, and biofilm-based growth systems. Ammonium may be applied gradually during any phase of reactor operation to either substitute for application in a single pulse or to supplement application in a single pulse. Alternating nitrogen sources may be combined with additional selection mechanisms, including reducing methane addition during PHB consumption, that may select for increased PHB production in methanotrophic cultures over time.

All such variations are considered to be within the scope and spirit of the present invention as defined by the following claims and their legal equivalents.

1-10. (canceled)

11. A method of producing PHB, comprising:
a. selecting, using a bioreactor, for a type II methanotroph in a biomass by exposing said biomass to ammonium and methane;
b. growing, in said bioreactor, said type II methanotroph in said biomass by exposing said biomass to nitrate and urea, nitrate and methane, or urea and methane;
c. accumulating, in said bioreactor, PHB from said type II methanotroph in said biomass in the absence of nitrogen and in the presence of methane, wherein a PHB-rich biomass is formed; and
d. harvesting said PHB from a portion of said PHB-rich biomass that is removed from said bio-reactor.

12. The method according to claim 11 further comprises repeating said selecting, said growing, said accumulating and said harvesting until a desired level of PHB production is achieved.

13. The method according to claim 11, wherein a reduced level of hydroxylamine relative to said ammonium is used instead of said ammonium to inhibit the growth of non-PHB producing organisms.

14. The method according to claim 11, wherein said growing is consecutively applied, using said bioreactor, with reduced frequency of ammonium growth steps.

15. The method according to claim 11, wherein methane is limited during said growing, wherein reduced methane levels increase ammonium toxicity and force organisms to draw upon stored said PHB, wherein and an increased production of said PHB during subsequent said accumulating occurs.

16. The method according to claim 11, wherein nitrogen sources are added incrementally throughout said selecting or said growing, wherein said nitrogen sources are selected from the group consisting of ammonium, nitrate, urea, and hydroxylamine.

17. The method according to claim 16, wherein said ammonium or hydroxylamine are added at incrementally reduced levels to increase selection for said type II methanotroph producers of said PHB.

18. The method according to claim 11, wherein said bioreactor comprises a biofilm growth system.
